(19) United States
(12) Plant Patent Application Publication
(10) Pub. No.: US 2018/0235128 P1
(43) Pub. Date: Aug. 16, 2018

(54) HOP PLANT NAMED 'J-007'

(50) ;

(71) Applicant: Nathan Jackson, Caldwell, ID (US)

(72) Inventor: Nathan Jackson, Caldwell, ID (US)

(21) Appl. No.: 15/530,713

(22) Filed: Feb. 16, 2017

(51) Int. Cl. A01H 6/00

(52) U.S. Cl. PLT/236

(57) ABSTRACT

A new hop plant named ‘J-007’ is disclosed. ‘J-007’ is characterized principally as to having a unique aroma, resistance to powdery mildew, compact laterals with a prolific cone set, and above average yield as compared with other aroma varieties.

### CROSS-REFERENCE TO RELATED APPLICATIONS

None

### STATEMENT REGARDING FEDERALLY SPONSORED RESEARCH OR DEVELOPMENT

None

Genus and species: Humulus lupulus.

Variety denomination: ‘J-007’.

### BACKGROUND OF THE INVENTION

‘J-007’ is a product of a controlled breeding program carried out by the inventor in the Treasure Valley in the State of Idaho. J-007 was one of several seedlings resulting from a controlled pollination cross in 2009 between female ‘Toyamadori’ and an unknown proprietary male hop plant owned by the inventor. A single plant of J-007 was identified in 2011, and observed during the growing seasons in 2012 and 2013. In 2014 that single plant of J-007 was asexually propagated by softwood cuttings, into a large scale test plot near Wilder, Id. for Idaho for further observation and evaluation in 2015 and 2016. An evaluation of that test plot shows that J-007 has been observed to retain its distinctive characteristics after asexual propagation, and has remained true to type.

### BRIEF DESCRIPTION OF THE PHOTOGRAPHS

FIG. 1 illustrates two rows of mature J-007 hop plant grown on a trellis;

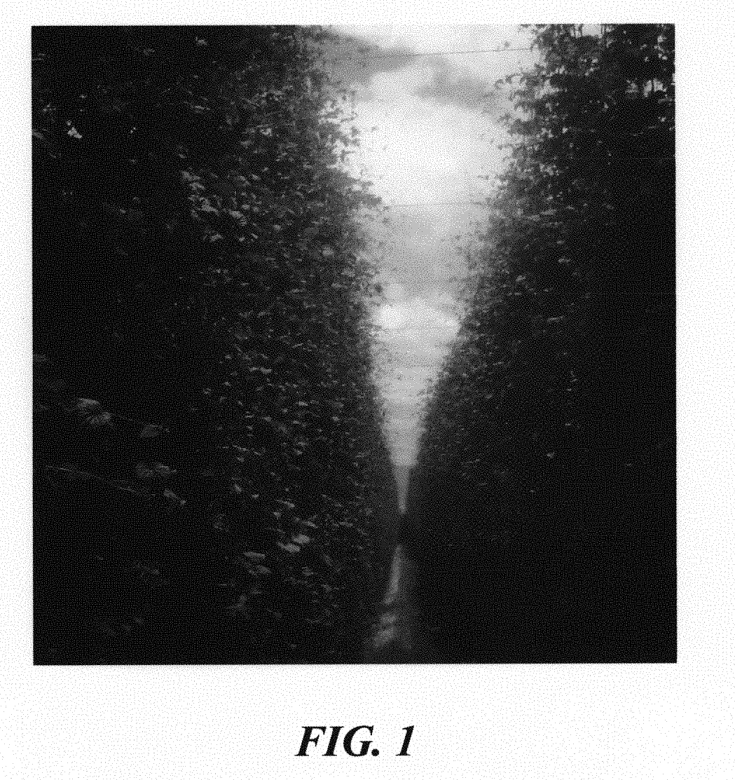

FIG. 2 illustrates a single mature whole cone growing on a J-007 hop plant;

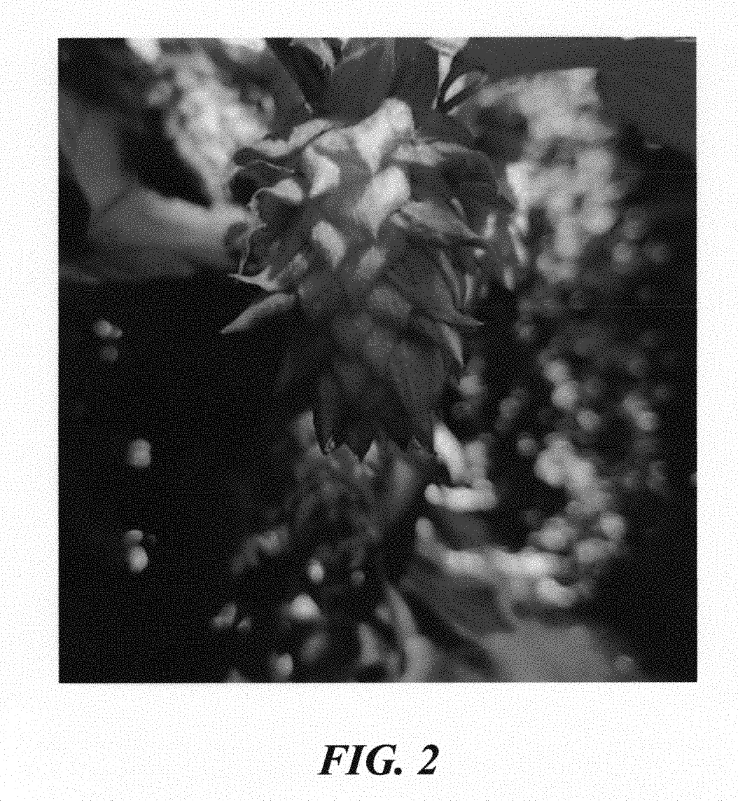

FIG. 3 illustrates the cones and leaves of a mature J-007 hop plant; and.

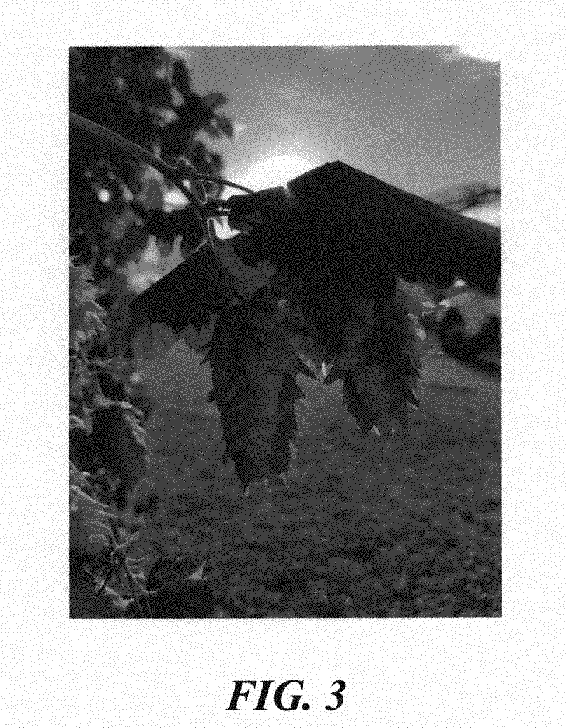

FIG. 4 illustrates a single mature whole cone of J-007 hop plant.

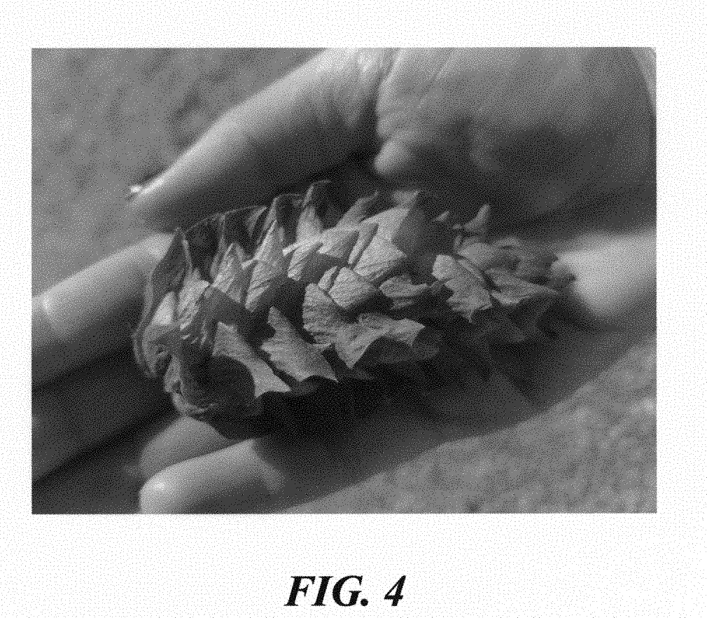

The colors of these illustrations may vary with lighting conditions and, therefore, color characteristics of this new variety should be determined with reference to the observations described herein, rather than from these illustrations alone.

### DETAILED BOTANICAL DESCRIPTION

The following description is based on observations made during the 2015 and 2016 growing seasons at Wilder, Id.

Table 1. sets forth some of the distinguishing characteristics of ‘J-007’ as compared to its female parent ‘Toyamadori’ and the ‘Summit’ variety:

TABLE 1

| Characteristic | ‘Toyamadori’ | ‘J-007’ | ‘Summit’ |
|---|---|---|---|
| Alpha Acid (α-Acids, % db) | 12.2 ± 0.6 | 12-14 | 18-19 |
| Beta Acids (β-Fraction, % db) | 7.2 ± 0.3 | 4.0-5.5 | 3.3-4.3 |
| Total Oil (ml/ 100 g) | 0.69 ± 0.19 | 1.2-2.4 | 1.5 |
| Myrcene (% of Total Oil) | 27 ± 5 | 46-58 | 48.5 |

It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants, of the new variety may vary from the stated average. The color chart used was The Royal Horticultural Society Colour Chart, Copyright 2004, and all color observations were made outdoors in full sunlight.
Ploidy: Diploid.
Use: Brewing beer.
Harvest date: September 20 to 30 during the 2015 and 2016 growing seasons, Wilder, Id.
Crop yield: 2300 to 2400 pounds per acre.
Disease susceptibility: Tolerant of hop downy mildew, powdery mildew, verticillium wilt, and the virus diseases found in U.S. hop growing areas.
Pest susceptibility: Average susceptibility to pests, including hop aphids and two spotted spider mites.
Plant shape: Fusiform when grown on a commercial hop trellis.
   Bine:
   Bine color.—Yellow Green 145A.
   Bine stripe.—Present.
   Bine stripe color.—Purple N77D.
   Bine internode length (at 180 cm (6′) high above the ground).—20-25 cm.
   Bine diameter (at 180 cm high (6′)).—12 mm to 14 mm.
   Bine average length.—487 cm to 580 cm (16′ to 19′).
Leaf:
   Leaf arrangement.—Opposite.
   Leaf shape.—Cordate.
   Mature leaf avg width.—18 cm.
   Mature leaf avg length.—16.5 cm.
   Leaf color, mature leaf upper surface.—Green N137B.
   Leaf color, mature leaf lower surface.—Green 147B.
   Number of leaf lobes.—3 to 5.
   Leaf venation.—Netted (palmately veined).
   Leaf vein color.—Yellow Green 144B.
   Leaf margin.—Moderately Dentate.
Petioles:
   Petiole avg length.—7 to 8 cm.
   Petiole color.—Green 143B.
   Petiole shape.—Channeled upper surface.
Laterals:
   Lateral avg length (at 180 cm (6′) high from the ground).—30 cm to 50 cm.
   Avg length of lateral internodes (at 180 cm (6′) high).—9 cm to 11 cm.
   Stipule position.—Downward.
Cones:
   Shape.—Ovoid to Elongated.
   Cone avg length.—4.5 cm to 5.0 cm.
   Cone avg diameter at shoulder.—2.0 cm to 2.5 cm.
   Cone avg weight.—250 mg to 320 mg.
   Compactness.—Moderately firm.
   Cone tip shape.—Tapered to a fairly distinct point.
   Strig.—Large with prominent pedicels.
   Lupulin gland color.—Ranging between Golden Yellow 2A to Yellow Green 150A (not distinctive of this variety).
   Lupulin gland shape.—Pedunculated oblong polyps.
   Aroma.—Apricot, nectarine, orange, and black tea.
Bracts:
   Bract tip shape.—Acute to acuminate.
   Bract tip position.—Mostly appressed with some bracts being slightly everted.
   Bract diameter.—13 mm to 14 mm.
Bracteoles:
   Bracteole diameter.—9 mm to 10 mm.
   Bracteole shape.—Lanceolate.
   Bracteole color.—Green 143C.
Analytical characteristics:
   Alpha acid.—12 to 14%.
   Beta acid.—4.0 to 5.5%.
   Cohumulone.—38 to 40% of alpha acids.
   Humulene.—10 to 15% of total oils.
   Caryophyllene.—4 to 8% of total oils.
   Myrcene.—46 to 55% of total oils.
   Farnesene.—<1% of total oils.
   Total oil.—1.2 to 2.4 m1/100 g.
   Hop storage index (hsi).—The cones of the present variety experience a 30% to 40% transformation of alpha acids after about 6 months of storage at 22° C.

What is claimed is:

1. A new and distinct hop plant as shown and described herein.
